(19) United States
(12) Patent Application Publication
(10) Pub. No.: US 2011/0312005 A1
(43) Pub. Date: Dec. 22, 2011

(54) Enzymatic modification of cell-surface H antigen by glycosyltransferases

(75) Inventors: Stephen Michael Henry, Auckland (NZ); Lissa Gwyneth Gilliver, Auckland (NZ); Cristina Simona Weinberg, Auckland (NZ)

(73) Assignee: Kode Biotech Limited, Auckland (NZ)

(21) Appl. No.: 13/137,096

(22) Filed: Jul. 20, 2011

Related U.S. Application Data
(63) Continuation of application No. 11/628,860, filed on May 21, 2007, now abandoned.

(30) Foreign Application Priority Data
Jun. 11, 2004 (NZ) 533480
Jan. 20, 2005 (NZ) 537826

(51) Int. Cl. C12Q 1/48

(52) U.S. Cl. 435/15

(57) ABSTRACT

This invention relates to cells with modified blood group antigen expression. In particular, the invention relates to cells for use in haematology, immunohaemotology and immunology assays as serology controls.

### CROSS-REFERENCE TO RELATED APPLICATIONS

This application is a continuation of application Ser. No. 11/628,860 filed May 21, 2007 which is a 371 of PCT/NZ2005/000126 filed Jun. 10, 2005 and claims priority to New Zealand Application Nos. 533480 filed 11 Jun. 2004, and 537826 filed Jan. 20, 2005, the entire contents of each of which are hereby incorporated by reference.

### ENZYMATIC MODIFICATION OF CELL-SURFACE H ANTIGEN BY GLYCOSYLTRANSFERASES

This invention relates to cells with modified blood group antigen expression. In particular, the invention relates to cells for use in haematology, immunohaemotology and immunology assays as serology controls.

### BACKGROUND

A critical function of blood centres is the testing of blood to accurately determine the blood group type of the individual from whom the blood (or other product) was obtained. Knowledge of the blood group type is essential for a variety of therapies including blood transfusion, organ transplantation, and the treatment of haemolytic diseases of the newborn. In particular, an individual's blood group type must be determined prior to being given a blood transfusion. A mismatch of blood group types can have disastrous consequences potentially leading to the death of the transfused individual.

The ABO blood group system represents the most important of the antigens on human red blood cells (RBCs) for blood transfusion serology. The phenotype of human RBCs belong to one of four major groups: A, B, AB, and O. The RBCs of each group respectively carry the A antigen, the B antigen, both A and B antigens, or neither. Antibodies are present in the blood against the blood group antigen which is absent from the RBCs. Thus, individuals of group A have anti-B, those of group B have anti-A, those of group O have anti-A and anti-B, and those of group AB have neither antibody. Before blood transfusion the blood must be cross-matched (either by testing the donor blood against the serum of the recipient or by matching the blood against records) to ensure that RBCs of one group are not given to an individual possessing antibodies against them.

RBCs are tested against reagents containing known antibodies (known as forward grouping) and serum is tested against RBCs possessing known antigens (known as reverse grouping).

Monoclonal antibodies (MAbs) have been used as blood typing reagents since the 1980's. When compared with traditional polyclonal antisera, monoclonal reagents offer increased specificity, consistent reactivity, and, in most cases, increased potency.

Routine quality control of blood group systems (for example, gel cards) and reagents is essential in any blood bank laboratory. Reagents and blood grouping systems may suffer reductions in specificity or potency during shipping, storage, or as a result of contamination during storage and use.

Monoclonal antibody reagents are required to identify all natural variations of ABO blood groups including subgroups of A and B. To ensure correct identification, monoclonal blood grouping reagents and blood grouping systems in blood bank laboratories should be tested against RBC serology controls (also referred to as “sensitivity controls” or “quality control cells”).

For this purpose, RBCs with a weak antigen expression are preferred as the serology control. This is because such RBCs can provide a better indication of an antiserum's potency for the identification of weak phenotypes.

There exist in nature various forms of weak or poorly expressing ABO subgroups. The level of NB antigen expression within each of the cell phenotypes is variable and generally unknown unless extensive analysis is performed.

Using RBCs of weak or poorly expressing ABO subgroups as serology controls is difficult in practice, due to the very low frequency of subgroup phenotype individuals. For example, the Ax phenotype is estimated as 0.003% of group A. Other subgroups have even lower frequency.

There is a compelling need in the industry for serology controls. The importance of this is magnified because there is general movement in pathology towards laboratories staffed by multi-skilled technicians who do not have extensive blood transfusion experience. ABO grouping reagents are some of the most regulated laboratory reagents, but they don't have adequate laboratory based serology controls for validation of the laboratory testing. Furthermore, the European Union in vitro devices directives state that laboratories “ . . . shall carry out the required controls and tests according to the latest state of the art.”

Currently, serological sensitivity of monoclonal antibody reagents (antisera) used for the detection of cells that poorly express carbohydrate antigens can be determined by one of several methods:
1. Testing of the antisera against RBCs of natural, weak or poorly expressing ABO subgroups.
   This involves finding a rare subgroup, preparing cells of this subgroup ready for use, and then using them as serology controls.
2. Testing of the antisera against RBCs expressing normal (common) levels of antigen.
   This does not give any indication of sensitivity.
3. Diluting the antisera prior to testing to determine potency.
   This involves diluting antibodies and testing against cells expressing normal (common) levels of antigen.

Testing in accordance with method 3 is the most common practice in the absence of serology controls expressing low levels of antigen.

Despite the different methods of measuring sensitivity, many laboratories simply rely on the quality control of the suppliers of testing reagents. Alternatively, laboratories may only batch test on a weekly or even monthly basis in the manner described for method 3 above.

As stated natural cells expressing low levels of antigen, due to their frequency, are very difficult to obtain and maintain supply. In addition, cells vary between individuals. Constant supply is difficult, if not impractical. Further, different populations have different frequencies of these weak subgroups.

Normal cells express high levels of antigen, for example in the region of >500,000 copies per red cell. When testing these cells, the reagents are typically diluted to show that at low dilution they can still react with RBCs and give a serologically positive result.

This dilution sensitivity method is time consuming. The results are then extrapolated to determine the detection level of antigen at normal dilution. This flawed methodology is unfortunately the practice in most laboratories.

Detection of reagent deterioration is only possible if regular time consuming dilution studies are undertaken or the reagents are tested against RBCs of weak subgroups.

Additional problems can occur with the dilution of antisera. Testing reagents are often biclonal and formulated to give specific performance characteristics. It is well known that the antibodies obtained from some clones are better than others at detecting ABO subgroups. As a consequence, reagents are often formulated as blends.

Dilution of such reagents negates their intrinsic performance features and thus will not reflect the true performance of the reagents. Furthermore, many testing reagents now come formulated for and pre-loaded into test card systems (i.e. gel cards) and thus cannot be tested by dilution methods.

Many laboratories do not presently routinely carry out sensitivity controlling of their ABO blood type testing reagents. Reports in the literature on the outcomes of accidental transfusion of a weak subgroup to an incompatible recipient indicates that these events are usually non-fatal.

Historically, a cross-match (the testing of the donor's blood against the recipient's serum) would detect an incompatibility between a weak subgroup mistyped and for transfusion to an incompatible recipient. However, these days cross-matching is not performed in many centres. Instead, correct blood typing of both the donor and recipient is relied upon.

It is therefore now more important that blood is accurately typed. The problem of not carrying out any testing against serology controls is that the blood type testing reagents may have deteriorated and a clinically significant subgroup may be incorrectly blood typed in the absence of cross-matching. Such blood may cause a mild to severe transfusion reaction.

There is a need for serology controls which have a known and predetermined level of antigen expression and are capable of being used for quality control of blood type testing reagents, such as monoclonal antibodies, and/or the calibration of testing systems to give accurate and standardised determinations of blood group types.

It is an object of this invention to provide serology controls for blood group type testing reagents and/or the calibration of testing systems, or to at least provide the public with a useful choice.

### STATEMENT OF INVENTION

In the first aspect the invention provides a serology control including cells comprising a derivative of an H-antigen molecule wherein the cell surface epitope of the antigen has been modified.

Preferably the cell surface epitope has been enzymatically modified.

Preferably the cell surface epitope is modified by the attachment of one or more monosaccharide units.

Preferably the monosaccharide units are selected from the group including galactose and N-acetylgalactosamine.

Preferably the monosaccharide units are alpha-linked.

In an embodiment of the invention the modified cell surface epitope is serologically equivalent to the cell surface epitope of A-antigen.

In an embodiment of the invention the derivative is serologically equivalent to A-antigen.

In an embodiment of the invention the modified cell surface epitope is serologically equivalent to the cell surface epitope of B-antigen.

In an embodiment of the invention the derivative is serologically equivalent to B-antigen.

Preferably the level of expression of modified cell surface epitope or derivative is serologically equivalent to the level of expression of A-/B-antigen by cells of a weak or poorly expressing ABO subgroup.

Preferably the level of expression of modified cell surface epitope or derivative is serologically equivalent to the clinically significant threshold for expression of A-/B-antigen by cells of an ABO blood group phenotype.

Preferably the cell is a red blood cell, more preferably a human red blood cell.

Preferably the level of expression of modified cell surface epitope or derivative is less than 5×10⁵ copies per cell, more preferably less than 1×10⁵ copies per cell, most preferably less than 2×10⁴ copies per cell, or the serological equivalent thereof.

Preferably the level of expression of modified cell surface epitope or derivative is greater than 1×10² copies per cell, more preferably more than 1×10³ copies per cell, or the serological equivalent thereof.

Preferably the modified cell surface epitope is serologically equivalent to the antigenic determinant of an immunodominant sugar, more preferably the immunodominant sugar of an A-antigen or B-antigen.

In a second aspect the invention provides a method of preparing a serology control by modifying the cell surface epitopes of a cell comprising the steps of:
contacting a solution of immunodominant sugar modifying enzyme and a solution of activated monosaccharide units with a suspension of cells; and
maintaining the suspension obtained at a temperature and for a time sufficient to allow modification of the cell surface epitope.

Preferably, the invention provides a method of preparing a serology control by modifying the cell surface epitope of an H-antigen molecule comprising the steps of:
contacting a solution of immunodominant sugar modifying enzyme and a solution of activated monosaccharide units with a suspension of H-antigen expressing cells; and
maintaining the suspension obtained at a temperature and for a time sufficient to allow modification of the cell surface epitope of the H-antigen molecule.

Preferably the modification of the cell surface epitope is by glycosylation.

Preferably the immunodominant sugar modifying enzyme is a glycosyltransferase, more preferably alpha-N-acetylgalactosaminyl transferase or alpha galactosyl transferase or a mixture of both.

Preferably the activated monosaccharide units are UDP-galactose, UDP-N-acetylgalactosamine, or a mixture of both.

Preferably the method provides a serology control including cells comprised of a derivative of an H-antigen molecule wherein the cell surface epitope of the antigen has been modified and is serologically equivalent to the epitope of an A- or B-antigen.

Preferably the method provides a serology control including cells comprised of a derivative of an H-antigen molecule wherein the derivative is serologically equivalent to an A- or B-antigen.

In an embodiment of the invention the activity of immunodominant sugar modifying enzyme is limiting for the rate of the modification.

In an embodiment of the invention the concentration of activated monosaccharide units is limiting for the rate of the modification.

Preferably the method includes the step of terminating the modification reaction, preferably by washing of the suspension obtained following maintaining the suspension at a temperature and for a time sufficient to allow modification.

In a third aspect the invention provides a serology control including cells prepared by the method of the second aspect of the invention, the cell consisting of a derivative of an H-antigen molecule wherein the cell surface epitope of the antigen has been modified.

Preferably the cell surface epitope has been enzymatically modified.

Preferably the cell surface epitope is modified by the attachment of one or more monosaccharide units.

Preferably the monosaccharide units are selected from the group including galactose and N-acetylgalactosamine.

Preferably the monosaccharide units are alpha-linked.

In an embodiment of the invention the modified cell surface epitope is serologically equivalent to the cell surface epitope of A-antigen.

In an embodiment of the invention the derivative is serologically equivalent to A-antigen.

In an embodiment of the invention the modified cell surface epitope is serologically equivalent to the cell surface epitope of B-antigen.

In an embodiment of the invention the derivative is serologically equivalent to B-antigen.

Preferably the level of expression of modified cell surface epitope or derivative is serologically equivalent to the level of expression of A-/B-antigen by cells of a weak or poorly expressing ABO subgroup.

Preferably the level of modified cell surface epitope expression is serologically equivalent to the clinically significant threshold for expression of A-/B-antigen by an ABO blood group phenotype.

Preferably the H-antigen expressing cells are red blood cells, more preferably human red blood cells.

Alternatively the H-antigen expressing cells are animal cells wherein H-antigen has been incorporated into the cell membrane in vitro.

Preferably the level of expression of modified cell surface epitope or derivative is less than 5×10⁵ copies per cell, more preferably less than 1×10⁵ copies per cell, most preferably less than 2×10⁴ copies per cell, or the serological equivalent thereof.

Preferably the level of expression of modified cell surface epitope or derivative is greater than 1×10² copies per cell, more preferably more than 1×10³ copies per cell, or the serological equivalent thereof.

Preferably the modified cell surface epitope is serologically equivalent to the antigenic determinant of an immunodominant sugar, more preferably the immunodominant sugar of an A-antigen or B-antigen.

In an embodiment of the first or third aspect of the invention the cells of the serology control are in suspension.

In an embodiment of the first or third aspect of the invention the cells of the serology control are localised to a surface.

Preferably the serology control contains a cell preservative (e.g. Alsevers™, Cellstab™, Celpresol™)

Preferably the serology control contains clinically significant antibodies to provide an additional control characteristic, more preferably the additional control characteristic is concurrent antibody control.

In a fourth aspect the invention provides a method for the determination of the sensitivity of a blood group type testing reagent including the steps of:
contacting the blood group type testing reagent with a serology control of either the first or third aspect of the invention; and
assessing the level of agglutination.

Preferably the assessing is by visual examination.

Preferably the method includes the step of determining the level of expression of modified cell surface epitope or derivative in the cell or cells of the serology control by reference to cells expressing known levels of antigen.

In a fifth aspect the invention provides a set or kit including two or more serology controls according to the first or third aspect of the invention.

Preferably the set or kit comprises serology controls including cells expressing the serological equivalent of group A and group B antigens. More preferably the set or kit comprises serology controls including red blood cells expressing the serological equivalent of group A, group B, Rh DCce (R1r) and Rh ce (rr) antigens. Most preferably the expression is at a level substantially equivalent to a clinically significant threshold.

The invention will now be described in detail.

### DETAILED DESCRIPTION

The inventors have established that A and B blood group antigens can be synthesised by the in vitro treatment of RBCs with glycosyltransferases. It is believed these enzymes add activated monosaccharides to H-antigen molecules incorporated in the cell membrane.

A range of cell surface epitopes that are serologically equivalent to the epitopes (glycotopes) of naturally occurring A- and/or B-antigens can be introduced or formed on the surface of the treated cells. The inventors have established that RBCs prepared by the method can be used as “serology controls” to assess the sensitivity of blood typing reagents (antisera)—in particular A and B antisera—and calibrate and validate testing systems.

Although it is preferred to modify the cell surface epitopes of human RBCs, the RBCs of other animals can be used. In addition, while the description refers principally to RBCs, it is to be appreciated that other cells such as platelets, white cells, plant cells, cell culture cells, bacterial cells and artificial cell membranes could be used.

Where the cells used do not naturally express H-antigen, the antigen may be incorporated into the cell membrane as a glycolipid by in vitro methods such as those described in international application PCT/NZ02/00214 (WO 03/034074) which is herein incorporated by reference.

Naturally occurring A and B blood group antigen molecules may be either glycolipids or glycoproteins. In the context of this description the term “cell surface epitope” is used to refer to the antigenic determinant of a cell membrane incorporated antigen expressed at the cell surface. The antigenic determinant or “epitope” of blood group antigens may also be referred to as a “glycotope”.

In the context of this description the term “serologically equivalent” means that the cells express antigen molecules with a modified cell surface epitope that provide a serological reaction equivalent to that of naturally occurring blood group antigen molecules.

It will be understood that the terms “H-antigen”, “A-antigen” and “B-antigen” refer to groups of antigen molecules that are serologically related to the extent that they are all blood group antigens. However, antigen molecules belonging to the same group are only serologically equivalent to the extent that the molecules may be grouped as “H-antigen”, “A-antigen” or “B-antigen” on the basis of their reaction, or lack thereof, with A and B antisera.

The antigens expressed by RBCs are characteristic of the blood group to which the RBCs belong. For example, the RBCs of blood group O express H-antigen and do not express A- or B-antigen.

All types of H-antigen terminate in a cell surface epitope that is fucose 1-2 linked to galactose. The derivatisation of the H-antigen molecule by glycosylation of this epitope results in a modified cell surface epitope that is serologically equivalent to the cell surface epitope of a naturally occurring A- or B-antigen molecule. The derivative of the H-antigen molecule is serologically equivalent to a naturally occurring A- or B-antigen when expressed in the membranes of RBCs.

As alluded to above, artificial H-antigens, i.e. antigens that do not naturally occur in a particular cell membrane, may be prepared and incorporated into the membrane of a cell, thereby providing an H-antigen expressing cell. The artificial H-antigen could, for example, be a synthetic glycolipid construct terminating in fucose 1-2 linked to galactose. Artificial A-antigen and B-antigen expressing cells of a variety of cell types can therefore be prepared.

The time for enzymatic synthesis of blood group antigens on the cell membranes of the cells depends on the relative concentrations of the enzyme solution and the availability of activated sugars. Additionally, the accessibility of the cell surface epitope of available H antigen is also a rate limiting step. Factors affecting enzymatic activity, including temperature, will also affect the density of the modified cell surface epitope synthesised on the cell surface.

The level of expression of modified cell surface epitope can be controlled by controlling the incubation conditions. The time of incubation and/or ratio of RBCs to enzyme may be controlled. Alternatively or in addition, the availability of activated sugars may be limited. A limited level of antigen expression can therefore be obtained by a range of methods employing different incubation conditions.

For the preparation of serology controls the conditions used are typically those that provide a serological result of approximately 2+. The actual value will be dependent on the sensitivity of the assay system used, for example, tile versus automation and the purpose for which the serology controls are to be used.

If cells expressing weaker levels of A or B antigen are desired, then lower concentrations of activated monosaccharide can be used. If strongly agglutinating phenotypes are desired, then higher concentrations of activated monosaccharide can be used. Alternatively controlling incubation times and/or enzyme concentrations in the presence of excess activated monosaccharide can bring about a similar result.

In addition to agglutination base assays, levels of glycotope expression can be determined by reference to cells expressing known levels of A- and/or B-antigen. Cells expressing known levels of A- and/or B-antigen can be prepared by the methods described in international application PCT/NZ02/00214 (WO 03/034074).

The introduction of blood group antigens exploits the principle that enzymes, such as glycosyltransferases, can specifically add activated sugars onto receptors. In the method, the H-antigens present in RBC membranes are the receptors. The glycosylation occurs without damaging non-target structures, e.g. proteins or carbohydrates with non-target linkages.

The inventors have recognised that RBCs expressing an enzymatically synthesised level of antigen, wherein the level of antigen expression is serologically equivalent to that of a naturally occurring ABO subgroup phenotype, provide particular advantages and benefits when used as serology controls.

The level of antigen expression for a serology control may be set at the clinically significant threshold at which failure to detect an antigen may result in a clinically significant transfusion reaction. The term “clinically significant threshold” is used to refer to the level of expression of an antigen below which a failure to detect the antigen will be of no clinical significance if transfused.

Other controls can be set at levels that will ensure confidence in the detection of weak subgroups. These controls can validate the performance of ABO blood grouping tests by making the sensitivity levels measurable. This can ensure the provision of safer ABO grouped blood.

Serology controls for use in transfusion medicine are made from group O cells where enzymes have synthesised specific amounts of A and/or B antigen, or their serological equivalent. The serology controls are used to give specific reaction scores in antigen detection assays. The assays may include tile, tube, gel card, and microplate methods, and any manual or automated platform which uses agglutination, or any other method of antigen detection (for example, enzyme linked immunoassay, flow cytometry etc).

Agglutination is one measure for antigen detection. Agglutination is the clumping of cells caused by antibody crosslinking antigens on different cells. Agglutination can be visualised manually (by eye) or in automated techniques by blood group analysers. Visualisation can be enhanced by using certain enzymes or by using radioactivity or fluorescence labels.

Manual agglutination reactions can be scored according to the following scheme:

| Agglutination Score | Observations |
|---|---|
| − | no clumps at all |
| (+) | indeterminant |
| + (i.e. 1+) | very small clumps |
| ++ (i.e. 2+) | several small clumps |
| +++ (i.e. 3+) | one large clump surrounded by small clumps |
| ++++ (i.e. 4+) | one single large clump |

The assessment of the level of agglutination may be by assessing direct agglutination or by assessing indirect agglutination where means of inducing agglutination are used, such as potentiation or using antiglobulin molecules.

An advantage of the invention is that as the amount of antigen detectable can be controlled to meet specific sensitivity requirements, one serology control could consist of red blood cells that give an agglutination score which correlates with a clinical significance level. Therefore, if this serology control produces a positive serology result then the user can be assured they will not miss any clinically significant subtypes.

Another serology control could consist of red blood cells obtained to express antigen at specific antigen thresholds, for example one for each of the different subtypes thereby allowing for known levels of sensitivity. Such serology controls could also be used to calibrate highly sensitive machines or could even be used in flow cytometry analysis for antigen quantitation curves.

Another advantage of the invention is that the methodology allows serology controls to be standardised and be consistent worldwide. This would allow comparisons of the performance of different laboratories and different methodologies. Inclusion of the cells in transfusion serology quality assurance programmes could set the ‘standard’ for the quality control of ABO blood group testing.

An embodiment of the invention may comprise a set or kit of serology controls comprising cells expressing group A (weak) phenotype and group B (weak) phenotypes. The set or kit could further comprise serology controls comprising cells expressing Rh DCce (R1r) and Rh ce (rr) control phenotypes. The set or kit could be used to ensure that both the ABO and RhD grouping reagents are quality controlled by the same set of serology controls.

Another set or kit comprising serology controls comprising cells with a range of weak A, B and AB phenotypes may be useful for more specialised laboratories.

The resuspending fluid used in conjunction with the serology controls may contain clinically significant antibodies.

Some laboratories perform ABO and RhD quality control effectively, but others do not. Some laboratories manufacture in-house suspensions of ABO and RhD quality control cells (A2B R1r, O rr). However, there is a degree of variation in these products because of blood donor phenotype heterogeneity.

The serology controls of this invention do not suffer this disadvantage because the weakened antigenic expression is precise, there is a lack of variability, and they can be readily prepared.

The invention will now be described by way of example only.

### EXAMPLE

Glycosyltransferase Enzyme

Synthetic recombinant analogues of human ABO(H) blood group glycosyltransferase glycosyltransferases, α3-N-acetylgalactosaminyltransferase (GTA) and α3-galactosyltransferase (GTB), were kindly supplied by Dr Monica Palcic of the University of Alberta, Canada (Seto et al. (1995) Eur. J. Biochem., 234; 323-328).
Group O RBCs were washed three times in saline, and packed after the final wash.
Three reactions were performed:
   GTA plus UDP-GalNAc;
   GTB plus UDP-Gal; and
   Control with no substrate or glycosyltransferase.
The glycosyltransferase was dissolved in Mops buffer (50 mM Mops buffer {pH 7}, 20 mM MnCl₂ {pH 7}, 1 mg/mL BSA).
The ingredients were added in the following manner: add water, buffer, NaCl₂ and substrate, then mix well, then add the cells followed by the glycosyltransferase to start the reaction. The final concentrations in the reaction mixture were: 50 mM Mops buffer (50 mM Mops buffer {pH 7}, 20 mM MnCl₂ {pH 7}, 1 mg/mL BSA), 150 mM NaCl, 60 μM substrate (UDP-GalNAc or UDP-Gal), 0.03 mU/μL glycosyltransferase (GTA or GTB) and MilliQ water in a total volume of 39.65 μL. This mixture was used to modify 26.35 μL washed packed RBCs.
For the controls Mops buffer was substituted for the glycosyltransferase and MilliQ water was substituted for the substrate.
The reaction mixture was incubated in a 37° C. waterbath for 2 hours and 40 min.
After this time the RBCs were washed three times in saline.
The RBCs were then tested for enzymatic modification by agglutination with the appropriate antibody in gel-cards.

TABLE 1
Antisera used for agglutination results for
all glycosyltransferase modified cells.

| | | | |
|---|---|---|---|
| Manufacturer | Catalogue ref | Batch number | Expiry date |

Anti-A

| | | | |
|---|---|---|---|
| BioClone, OCD | Experimental reagent | 01102 | — |

Anti-B

| | | | |
|---|---|---|---|
| BioClone, OCD | Experimental reagent | 01103 | — |

TABLE 2
Diamed results of group O RBCs transformed by building the
A and/or B antigens using A and B glycosyltransferase glyco-
syltransferases. The glycosyltransferase and substrate were in
excess. Cells were tested against Bioclone antisera.

| | | GTA | | | | GTB | | | |
|---|---|---|---|---|---|---|---|---|---|
| | | 0.03 | | | | 0.03 | | | |
| | | mU/μL | | 0 | | mU/μL | | 0 | |
| | | Antisera | | | | | | | |
| Substrate | Amount | A | B | A | B | A | B | A | B |
| UDP-GalNAc | 60 mM | 4+ | 0 | 0 | | 0 | 1+ | | |
| (A) | 0 | | | | | | | | |
| UDP-Gal (B) | 60 mM | 3+ | 0 | | | 0 | 4+ | | 0 |
| | 0 | | | | | | | | |

The glycosyltransferases were shown to effectively modify group O RBCs to A or B.

Whilst not wishing to be bound by theory it is believed that the A and B blood group antigens are constructed on the pre-existing H antigens of the group O RBCs. Incubation of the glycosyltransferases with the non-complementary substrate (eg GTB with UDP-GalNAc) was undertaken to assess the absolute specificity of the two glycosyltransferases.

Neither of the glycosyltransferases appear to be able to utilise the non-complementary nucleotide donor monosaccharide (eg GTB and UDP-GalNAc) when visualised in an agglutination test using the non-complementary antibody (eg GTB and anti-A).

Surprisingly, positive reactions were seen using the glycosyltransferases with the non-complementary nucleotide donor monosaccharide (eg GTB with UDP-GalNAc) when visualised in an agglutination test using the complementary antibody (eg GTB and anti-B).

GTA with the UDP-Gal substrate gave a 3+ agglutination score against anti-A, while GTB with UDP-GalNAc gave a 1+ agglutination score against anti-B in Diamed cards.

This implies that the glycosyltransferases were able to add the complementary monosaccharide (eg GTA was adding UDP-GalNAc, and GTB was adding UDP-Gal) even though the substrate reagent was supposed to contain the non-complementary monosaccharide. This is possibly attributed to some contamination of the substrates with other nucleotide donor monosaccharides, i.e. UDP-GalNAc probably contains some UDP-Gal and vice versa.

It is interesting that the strength of these reactions is so different—the GTA reaction is stronger than the GTB reaction. Given that GTB is active at higher dilutions than GTA (see Table 5) and at lower concentrations of substrate than GTA (see Table 3 and Table 4), it would seem reasonable to expect that GTB would show a stronger reaction with trace UDP-Gal than would GTA with trace UDP-GalNAc.

One possible explanation for this is the level of contamination of the substrates. The catalogue for these reagents states a 98% purity for the UDP-GalNAc, but only a 95% purity for the UDP-Gal. In addition, it has been reported that wild-type GTA can utilise the B donor (UDP-Gal) with three times greater efficiency than wild-type GTB can use the A donor (Seto et al. (1999) Eur. J. Biochem., 259; 770-775).

The apparent reason for this is the poor binding ability of GTB towards UDP-GalNAc because the large N-acetyl group on carbon 2 is not easily fitted into the space designed to accommodate only the small hydroxyl group of Gal.

Nucleotide Donor Monosaccharide Dilutions

Dilutions of the nucleotide donor monosaccharides (UDP-GalNAc and UDP-Gal) were tested in the range of 1:10 to 1:100000 (results of lower dilutions not shown), against excess quantities of the respective, i.e. complementary, glycosyltransferases.

UDP-N-Acetylgalactosamine

TABLE 3
Diamed results of glycosyltransferase synthesis of A antigens on
group O RBCs with dilutions of the substrate. The glycosyltransferase
was in excess. Cells were tested against Bioclone
anti-A. Controls contained glycosyltransferase, but no substrate.

| | Substrate dilutions | | | | | | | |
|---|---|---|---|---|---|---|---|---|
| Substrate | 1 | 1:5 | 1:10 | 1:20 | 1:40 | 1:60 | 1:80 | 0 |
| UDP-GalNAc | 4+ | | 4+ | | | | | 0 |
| | | | 1+ | vw | 0 | 0 | 0 | |
| | 4+ | 2-3+ | 1+ | 0 | | | | |

UDP-Galactose

TABLE 4
Diamed results of glycosyltransferase synthesis of B antigens on
group O RBCs with dilutions of the substrate. The glycosyltransferase
was in excess. Cells were tested against Bioclone
anti-B. Controls contained glycosyltransferase, but no substrate.

| | Substrate dilutions | | | | | | | | |
|---|---|---|---|---|---|---|---|---|---|
| Substrate | 1 | 1:10 | 1:100 | 1:200 | 1:400 | 1:600 | 1:800 | 1:1000 | 0 |
| UDP-Gal | 4+ | 4+ | 4+ | | | | | 0 | 0 |
| | | | 4+ | 3+ | 2-3+ | 2+ | 1+ | | |

Glycosyltransferase Dilutions

Glycosyltransferase (GTA and GTB) dilutions of 1:2, 1:4, 1:8, 1:16 and 1:32 plus GTB dilutions of 1:64, 1:128, 1:256, 1:512, 1:1024 and 1:2168 were tested against excess quantities of the respective, i.e. complementary, substrate.

TABLE 5
Diamed results of glycosyltransferase synthesis of A and B antigens on group O
RBCs with dilutions of the glycosyltransferase. The substrate was in excess. Cells were
tested against the relevant Bioclone antisera. Controls contained substrate, but no
glycosyltransferase.

| | Glycosyltransferase dilutions | | | | | | | | | | | | |
|---|---|---|---|---|---|---|---|---|---|---|---|---|---|
| Enz | 1 | 1:2 | 1:4 | 1:8 | 1:16 | 1:32 | 1:64 | 1:128 | 1:256 | 1:512 | 1:1024 | 1:2168 | 0 |
| A | 4+ | 4+ | 3+ | 0 | 0 | 0 | | | | | | | 0 |
| B | 4+ | 4+ | 4+ | 4+ | 4+ | 4+ | 4+ | 4+ | 4+ | 2-3+ | 2+ | 0 | 0 |

Glycosyltransferase and Nucleotide Donor Monosaccharide Interaction

To understand the dynamics and performance of GTA and GTB multiple glycosyltransferase and substrate combinations were tested. GTA was used neat, while GTB was used at a 1:200 dilution. The substrates were added in excess.

TABLE 6
Diamed agglutination results of cells transformed with
combinations of GTA, GTB, UDP-GalNAc and UDP-Gal.

| | GTA | | | GTA + GTB | GTB | | |
|---|---|---|---|---|---|---|---|
| | | GalNAc + | | GalNAc + | | GalNAc + | |
| Anti | GalNAc | Gal | Gal | Gal | GalNAc | Gal | Gal |
| A | 4+ | 0 | 3+ | 0 | 0 | 0 | |
| | 4+ | | | | | | |
| B | | 0 | 0 | 4+ | 1+ | 4+ | 0? |
| | | | | | | | 4+ |

These results show that the presence of UDP-GalNAc did not affect the ability of GTB to make B epitopes on group O RBCs. However, the presence of UDP-Gal appears to prevent GTA from catalysing the postulated transfer of GalNAc to H antigens on group O RBCs. This is in contrast to the 3+ agglutination score in the reaction against anti-A seen when GTA was incubated with UDP-Gal.

In a previous experiment (see Table 2), the presence of major quantities of UDP-Gal did not interfere with the proposed utilisation of the contaminating UDP-GalNAc. Some other explanation may therefore be indicated.

Stability Trials

Trials were performed to assess the stability of enzyme modified RBCs in terms of physical condition (determined by cell colour and haemolysis levels) and antigen expression (measured by agglutination with the relevant antisera).

GTA-Modified Cells

TABLE 7
Stability trial of GTA modified group O RBCs.

| | GTA dilutions | | | | | | | | | | | | | | |
|---|---|---|---|---|---|---|---|---|---|---|---|---|---|---|---|
| | 1 | | | 1:2 | | | 1:3 | | | 1:4 | | | 1:5 | | |
| Day | A | CS | CP | A | CS | CP | A | CS | CP | A | CS | CP | A | CS | CP |
| 1 | 4+ | | | 4+ | | | 4+ | | | 4+ | | | 4+ | | |
| 3 | 4+ | 4+ | 4+ | 3− | 4+ | 4+ | 2− | 3+ | 3+ | 3+ | 3+ | 3+ | 3+ | 3+ | 3+ |
| | | | | 4+ | | | 3+ | | | | | | | | |
| 9 | 4+ | 4+ | 4+ | 4+ | 4+ | 4+ | 2− | 2− | 2− | 2− | 2− | 3+ | 2− | 2− | 2− |
| | | | | | | | 3+ | 3+ | 3+ | 3+ | 3+ | | 3+ | 3+ | 3+ |
| 13 | 4+ | 4+ | 4+ | 4+ | 4+ | 4+ | 3+ | 3− | 3+ | 3+ | 3− | 3+ | 3+ | 3− | 3+ |
| | | | | | | | | 4+ | | | 4+ | | | 4+ | |
| 16 | 4+ | 4+ | 4+ | 4+ | 4+ | 4+ | 2− | 3+ | 2− | 3+ | 3+ | 2− | 2− | 3+ | 2− |
| | | | | | | | 3+ | | 3+ | | | 3+ | 3+ | | 3+ |

Cells were tested immediately after transformation on day 1 before being suspended in one of the three cell preservative solutions (A—Alsevers™; CS—Cellstab™; and CP—Celpresol™). Thereafter, cells were tested from these cell preservative solutions.

GTB-Modified Cells

TABLE 8
Stability trial of GTB modified group O RBCs.

| | GTB dilutions | | | | | | | | | | | | | | |
|---|---|---|---|---|---|---|---|---|---|---|---|---|---|---|---|
| | 1:64 | | | 1:128 | | | 1:256 | | | 1:512 | | | 1:1024 | | |
| Day | A | CS | CP | A | CS | CP | A | CS | CP | A | CS | CP | A | CS | CP |
| 1 | 4+ | | | 4+ | | | 4+ | | | 4+ | | | 4+ | | |
| 3 | 4+ | 4+ | 4+ | 4+ | 4+ | 4+ | 4+ | 4+ | 4+ | 4+ | 4+ | 4+ | 3− | 3− | 3− |
| | | | | | | | | | | | | | 4+ | 4+ | 4+ |
| 9 | 4+ | 4+ | 4+ | 4+ | 4+ | 4+ | 4+ | 4+ | 4+ | 4+ | 4+ | 4+ | 4+ | 4+ | 4+ |
| 13 | 4+ | 4+ | 4+ | 4+ | 4+ | 4+ | 4+ | 4+ | 4+ | 4+ | 4+ | 4+ | 4+ | 4+ | 4+ |
| 16 | 4+ | 4+ | 4+ | 4+ | 4+ | 4+ | 4+ | 4+ | 4+ | 4+ | 4+ | 4+ | 3− | 3− | 3− |
| | | | | | | | | | | | | | 4+ | 4+ | 4+ |

Cells were tested immediately after transformation on day 1 before being suspended in one of the three cell preservative solutions.

GTA/GTB-Modified Cells

A two-step block titre of GTA and GTB dilutions with excess substrate was performed. The GTA/UDP-GalNAc incubation was carried out first, and the cells were washed before the GTB/UDP-Gal incubation was done.

The GTB 1:400 (against all GTA dilutions) agglutination results were obtained on the day of transformation (day 1), while the other initial results were obtained on the next day (day 2). Increase agglutination scores were obtained for the GTA modified cells after overnight storage. (As the GTB results gave the maximum 4+ agglutination on day 1 any increase was not detectable.)

These results show a trend that appears to indicate an interaction between the two antigens postulated to be constructed on the pre-existing H antigens of the enzyme modified group O RBCs. Across all the dilutions of GTA (although most noticeable at the 1:4 dilution), the strength of agglutination with anti-A is weaker with the lower dilutions of GTB (1:400). As the dilution of GTB increases, so does the strength of the agglutination of the enzyme modified RBCs with anti-A.

TABLE 9
Stability of cells transformed by a two-step block titre of GTA and GTB dilutions with
excess substrate. Agglutinations performed in Diamed gel cards against Bioclone antisera.
Cells were stored in Celpresol ™.

| | | GTB | | | | | | | |
|---|---|---|---|---|---|---|---|---|---|
| | | 1:400 | | 1:600 | | 1:800 | | 0 | |
| GTA | Day | Anti-A | Anti-B | Anti-A | Anti-B | Anti-A | Anti-B | Anti-A | Anti-B |
| 1:2 | 1 | 3+ | 4+ | | | | | | |
| | 2 | | | 4+ | 4+ | 4+ | 4+ | 4+ | 0 |
| | 4 | 4+ | 4+ | 4+ | 4+ | 4+ | 4+ | 4+ | 0 |
| | 7 | 3+ | 4+ | 3-4+ | 4+ | 3-4+ | 4+ | 3-4+ | 0 |
| | 14 | 3+ | 4+ | 4+ | 4+ | 4+ | 4+ | 4+ | 0 |
| | 21 | 3+ | 4+ | 3-4+ | 4+ | 3-4+ | 4+ | 3-4+ | 0 |
| 1:3 | 1 | 2-3+ | 4+ | | | | | | |
| | 2 | | | 4+ | 4+ | 4+ | 4+ | 4+ | 0 |
| | 4 | 3+ | 4+ | 3-4+ | 4+ | 4+ | 4+ | 4+ | 0 |
| | 7 | 2-3+ | 4+ | 3+ | 4+ | 3+ | 4+ | 3+ | 0 |
| | 14 | 2-3+ | 4+ | 3-4+ | 4+ | 3+ | 4+ | 3+ | 0 |
| | 21 | 2-3+ | 4+ | 3-4+ | 4+ | 3-4+ | 4+ | 3-4+ | 0 |
| 1:4 | 1 | 0 | 4+ | | | | | | |
| | 2 | | | 3+ | 4+ | 3+ | 4+ | 3+ | 0 |
| | 4 | 2+ | 4+ | 3+ | 4+ | 3+ | 4+ | 3+ | 0 |
| | 7 | 1-2+ | 4+ | 3+ | 4+ | 3+ | 4+ | 3+ | 0 |
| | 14 | 1+ | 4+ | 3+ | 4+ | 3+ | 4+ | 3+ | 0 |
| | 21 | 1+ | 4+ | 3+ | 4+ | 3+ | 4+ | 3+ | 0 |
| 0 | 1 | 0 | 0 | | | | | | |
| | 2 | | | 0 | 4+ | 0 | 4+ | 0 | 0 |
| | 4 | 0 | 4+ | 0 | 4+ | 0 | 4+ | 0 | 0 |
| | 7 | 0 | 4+ | 0 | 4+ | 0 | 4+ | 0 | 0 |
| | 14 | 0 | 4+ | 0 | 4+ | 0 | 4+ | 0 | 0 |
| | 21 | 0 | 4+ | 0 | 4+ | 0 | 4+ | 0 | 0 |

The explanation that the lower dilutions of GTB were so efficient that they “consumed” most of the H antigen acceptors is inapplicable because the group O RBCs were exposed to GTA first—the GTA reaction was performed before the GTB reaction. It would seem most likely that the opposite would be true—that the B agglutinations would be affected by the GTA dilutions i.e. that the B agglutinations would be weaker with the lower GTA dilutions.

It is important to note that this phenomenon could also be occurring with the B antigens, but may be obscured by the maximum 4+ score that all dilutions of GTB have produced.

Two factors preclude the drawing of any conclusions about the basis of these agglutination score trends. Although agglutination is directly related to the amount of antigen expression, it is also significantly affected by the shape of the RBCs and the nature of antigen presentation.

Additionally, the precursor specificities of the glycosyltransferases are unknown, and GTA may be catalysing the addition of GalNAc to H acceptors of different structure than the ones GTB is able to utilise. These structural differences may encompass, among others, variations in anchor molecule (protein or lipid), size of sugar chain (5 sugars up to polyglycosylceramides which are >100), sugar chain core type or terminal type (type 1, 2, 3 etc.).

Antisera Comparison

Comparison of the performance of a panel of historical antisera was conducted using natural AB RBCs and enzyme modified group O RBCs expressing both A and B antigens.

The cells were first modified with GTA at a dilution of 1:2, and then with GTB at a dilution of 1:800 (see Table 9 for the results of these cells in other testing). These cells had been enzyme modified 12 days prior to undertaking this antisera comparison, and had been stored in Celpresol at 4° C.

TABLE 10
Expired A monoclonal blend antisera used in
the comparison trial (Table 11).

| A Antisera | Catalogue | Batch | |
|---|---|---|---|
| Manufacturer | ref | number | Date of Exp or Manuf. |
| Albaclone, SNBTS | 801320100 | Z0010680 | Exp 01.02.03 |
| Bioclone, OCD | Experimental | 01102 | Manuf 16.05.02 |
| Bio Labs | — | 8636 | Exp 10.87 |
| Epiclone, CSL | — | 20901 | Exp 11.93 |
| Gamma Clone | — | AM30-1 | Exp 19.07.93 |
| Immucor | — | 1A6137A | Exp 22.04.93 |
| Lorne Labs | 600010 | 60086D | Exp 08.01 |
| Novaclone, Dominion | — | NA00503 | Exp 08.05.93 |
| Organon | — | 112Z15A | Exp 19.12.93 |
| Seraclone, Biotest (1) | 801320100 | 132051 | Exp 29.05.93 |
| Seraclone, Biotest (2) | 801320100 | 1310401 | Exp 12.04.03 |

TABLE 11
Diamed results of agglutination of AB RBCs, natural and enzyme
modified, against a panel of historical A antisera (see Table 10).

RBCs

| | | Glycosyltransferase |
|---|---|---|
| A Antisera | Natural AB | modified AB |
| Albaclone, SNBTS | 4+ | 4+ |
| Bioclone, OCD | 4+ | 4+ |
| Bio Labs | 3+ | 1-2+ |
| Epiclone, CSL | 3+ | 1-2+ |
| Gamma Clone | 2-3+ | 0 |
| Immucor | 2-3+ | 0 |
| Lorne Labs | 3-4+ | 3+ |
| Novaclone, Dominion | 3+ | 1-2+ |
| Organon | 2-3+ | 1+ |
| Seraclone, Biotest (1) | 4+ | 4+ |
| Seraclone, Biotest (2) | 4+ | 4+ |

TABLE 12
Expired B monoclonal blend antisera used
in the comparison trial (Table 13).

| B Antisera | | Batch | |
|---|---|---|---|
| Manufacturer | Catalogue ref | number | Date of Exp or Manuf. |
| Albaclone, SNBTS | — | Z0110600 | Exp 27.04.03 |
| Bioclone, OCD (1) | Developmental | 01103 | Manuf 16.05.02 |
| Bioclone, OCD (2) | — | BBB589A | Exp 21.11.99 |
| Bio Labs | — | 8625 | Exp 07.87 |
| Epiclone, CSL (1) | BO266 | 23801 | Exp 05.00 |
| Epiclone, CSL (2) | BO266 | 20801 | Exp 11.93 |
| Immucor | — | IE6240-1 | Exp 22.05.93 |
| Lorne Labs | 610010 | 61003A | Exp 08.01 |
| Meditech | — | 110199 | Exp 11.01 |
| Organon | — | 112X19B | Exp 16.12.93 |
| Seraclone, Biotest | 801345100 | 114061 | Exp 12.06.93 |

TABLE 13
Diamed results of agglutination of AB RBCs, natural and enzyme
modified, against a panel of historical B antisera (see Table 12).

RBCs

| | | Glycosyltransferase |
|---|---|---|
| B Antisera | Natural AB | modified AB |
| Albaclone, SNBTS | 4+ | 4+ |
| Bioclone, OCD (1) | 4+ | 4+ |
| Bioclone, OCD (2) | 4+ | 4+ |
| Bio Labs | 4+ | 4+ |
| Epiclone, CSL (1) | 4+ | 2+ |
| Epiclone, CSL (2) | 3-4+ | 0 |
| Immucor | 4+ | 3+ |
| Lorne Labs | 4+ | 3+ |
| Meditech | 4+ | 4+ |
| Organon | 4+ | 4+ |
| Seraclone, Biotest | 4+ | 4+ |

All the antisera can detect the A and B antigens on the natural AB cells, but some show a reduced ability or are completely unable to detect the antigens on the enzyme modified cells.

Although the invention has been described in detail with reference to specific examples, it should be appreciated that variations and modifications may be made without departing from the scope of the claims. Furthermore, where known equivalents exist to specific features, such equivalents are incorporated as if specifically referred in this specification.

1-65. (canceled)

66. A method for the determination of the sensitivity of blood group type testing reagents including the steps of:
a. contacting the blood group testing reagents with a serology control; and
b. assessing the level of agglutination,
where the serology control comprises human red blood cells prepared by a method of modifying the cell surface epitope of an H-antigen molecule of a cell by glycosylation comprising the steps of:
a. contacting a solution of glycosyltransferase and a solution of activated monosaccharide units with a suspension of H-antigen expressing cells; and
b. maintaining the suspension obtained at a temperature and for a time sufficient to allow modification of the cell surface epitope,
where the level of modified cell surface epitope expression is serologically equivalent to the clinically significant threshold for expression of A- and/or B-antigen.

67. The method according to claim 66 where the glycosyltransferase is an α-N-acetylgalactosaminyl transferase, α-galactosyl transferase or a mixture of both, and the activated monosaccharide units are UDP-galactose, UDP-N-acetylgalactosamine, or a mixture of both.

68. The method according to claim 66 where the activity of glycosyltransferase is limiting for the rate of the modification.

69. The method according to claim 66 where the concentration of activated monosaccharide units is limiting for the rate of the modification.

70. The method according to claim 66 where the method includes the step of terminating the modification reaction.

71. The method according to claim 70 where the step of terminating the modification reaction is by washing of the suspension obtained following maintaining the suspension at a temperature and for a time sufficient to allow modification.

72. The method according to claim 66 where the cells of the serology control are in suspension.

73. The method according to claim 66 where the cells of the serology control are localised to a surface.

74. A set or kit including two or more serology controls for use in the method according to claim 66.

75. A standardised method of confirming the sensitivity of ABO blood group type testing reagents and systems comprising the steps of:
Contracting the reagents or systems with a serology control; and
Determining the level of agglutination;
where the serology control comprises O-group red blood cells glycosylated in vitro to provide cells expressing a level of A- and/or B-antigen serologically equivalent to the clinically significant level of expression of the antigen.
